(19) United States
(12) Patent Application Publication
(10) Pub. No.: US 2024/0225970 A1
(43) Pub. Date: Jul. 11, 2024

(54) A METHOD OF REDUCING SKIN IRRITATION, COMPRISING THE APPLICATION TO THE SKIN OF AN ALPHA-BISABOLOL PREPARED BY A BIOTECHNOLOGICAL PROCESS

(71) Applicant: Givaudan SA, Vernier (CH)

(72) Inventors: Marie MEUNIER, Les Petites Loges (FR); Emmanuel AUSSANT, Paris (FR); Romain Reynaud, Toulouse (FR); Amandine Scandolera, Reims (FR)

(21) Appl. No.: 18/011,018

(22) PCT Filed: Jul. 7, 2021
(86) PCT No.: PCT/EP2021/068897

§ 371 Date: Dec. 16, 2022

(30) Foreign Application Priority Data
Jul. 7, 2020 (GB) 2010390.9

(51) Int. Cl. A61K 8/34; A61K 8/02; A61Q 19/00; C07C 33/14; C11D 3/20; C11D 3/50; C12P 7/02; D01F 1/10; D06M 13/144

(52) U.S. Cl. CPC A61K 8/34; A61K 8/0204; A61Q 19/005; C07C 33/14; C11D 3/2037; C11D 3/505; C12P 7/02; D01F 1/10; D06M 13/144; D06M 2200/00

(57) ABSTRACT

A method of reducing skin irritation, comprising the application to the skin of an α-bisabolol prepared by a biotechnological process. The α-bisabolol thus prepared provides a substantial reduction in irritation, especially in fabrics worn for a prolonged period.

This disclosure relates to bisabolol and to its use as a soothing agent, particularly on wearable fabrics.

Wearable fabrics in the form of garments need regular washing in order to maintain their cleanliness and freshness. This can present problems when the fabrics have direct contact with the skin (the use of which term also includes the scalp). Laundry products such as laundry detergents and fabric conditioners of necessity contain ingredients that can be harsh to skin and scalp, for example, surfactants. While the majority of wearers experience little or no discomfort on prolonged wearing with direct skin contact, others with more sensitive skin can experience noticeable and even considerable discomfort and, in extreme cases, rashes and eczema. This is especially problematic where a garment is in proximity to the skin for a long period, and/or is covering a large area. One particular instance is any form of feminine Islamic head covering.

α-bisabolol

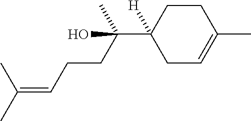 [SMILES: CC(C)=CCC[C@@](C)(O)[C@@H]1CC=C(C)CC1]

6-methyl-2-(4-methylcyclohex-3-en-1-yl)hept-5-en-2-ol (CAS 23089-26-1) is a natural monocyclic sesquiterpene alcohol. It is a component of chamomile and lavender oils, the richest source being the New Caledonian shrub Myoporum crassiforum, whose oil has up to 65% of α-bisabolol. The current major source is the Brazilian candeia tree (Eremanthus erythropappus) by distillation or by chemical synthesis.

The soothing and healing properties of α-bisabolol are well known and have been widely used in cosmetics and pharmaceutical products. It is also known to add α-bisabolol to face masks (see, for example, Chinese publication CN110251420). It has been observed that α-bisabolol in combination with ginger root extract applied to fabric can have a soothing effect when applied to fabric (International Publication WO 2021/073775). This blend may be used in encapsulated form.

It has now been found that α-bisabolol produced by a particular method can result in a surprising improvement in irritation reduction over bisabolols produced synthetically or from plant extracts. There is therefore provided a method of reducing skin irritation, comprising the application to the skin of an α-bisabolol prepared by a biotechnological process.

There is also provided the use of α-bisabolol prepared by a biotechnological process in the reduction of skin irritation by means of its application to the skin.

There is also provided the use of α-bisabolol prepared by a biotechnological process in the reduction of skin irritation caused by prolonged contact with a fabric, comprising its incorporation into the fabric in an irritation-reducing proportion.

There is also provided a fabric washed with a laundry product and having a reduced potential for causing skin irritation on prolonged contact, the fabric having been washed using a laundry product comprising an irritation-reducing proportion of α-bisabolol prepared by a biotechnological process.

By “application to the skin” is meant any form of application, be it in the form of skin application product, such as a cosmetic, personal care or hair care composition, for example, a cream or a salve, a shampoo or hair conditioner. It may also be the application of a fabric to the skin, such as wearing an item of clothing, in which the α-bisabolol is incorporated, by, for example, direct incorporation into the fabric itself, or by the fabric having been washed in a laundry composition comprising the α-bisabolol.

By “biotechnological process” is meant any process involving the use of a living organism, such as a prokaryotic microorganism such as a bacterium or a eukaryotic microorganism such as a yeast, in the production. Such methods may include a fermentation step, involving the growth of microorganisms in a growth medium. They may also include a bioconversion step, that is, an enzyme-catalysed process in which an intermediate or a precursor is converted into a desired end-product. They may also include a biosynthesis step, in which a substrate is converted to a more complex product my means of enzyme catalysis. Any such process is useful in the method of this disclosure.

It has been surprisingly found that α-bisabolol produced by a biotechnological method (hereinafter “bio-bisabolol”) is particularly effective in providing reduced skin irritation and long-lasting relief therefrom. It is believed, without limiting the scope of this disclosure in any way, that such methods produce a high proportion of the desirable (−)-α-bisabolol, free from the undesirable enantiomers that are produced by synthetic processes, or that are present in the products derived from plants.

Any microorganism capable of producing bio-bisabolol may be used. An example of such a microorganism may be found in Korean Patent No. 101958113, which utilises an improved Escherichia genus strain. In Microb. Cell Fact (2016) 15:185, there is described the use of a metabolically-engineered E. coli in a fermentation production of α-bisabolol.

A particular example of biotechnological production is one using Saccharomyces cerevisiae (baker's yeast) in a culture medium, using the method described in Biochem. J. (2014) 463, 239-248. This method, when followed by a distillation step, gives a bio-bisabolol of at least 99% purity. Therefore, in a particular embodiment, there is provided a bio-bisabolol of at least 99% purity, produced by this method.

By “laundry product” is meant any product that is used in the washing of fabrics, typically laundry detergents (both liquid and powder), fabric conditioners and the like. All of the usual commercially-available products may be used, containing all of the standard ingredients generally used in art-recognised proportions in such products.

The bio-bisabolol may be added to the product simply by mixing it into the product, or it may be added dissolved in a suitable medium.

In a particular embodiment, the bio-bisabolol is added in encapsulated form. Encapsulation has been typically used in laundry products for the encapsulation of fragrance, both to preserve the fragrance components from potential degradation in a harsh environment and to allow for delayed release. In the case of bio-bisabolol, this may be encapsulated in pure form (it is a viscous oil), or it may be dissolved in a suitable solvent, typically a terpene. It may be used alone, or it may be incorporated into a fragrance formulation, which is then encapsulated.

Any suitable encapsulation method may be used, depending on its manner of intended utilisation. One such method is the dispersion of the α-bisabolol either pure or in solution in a starch solution, followed by the formation of granules by spray drying into a heated atmosphere. It may also be encapsulated in one of the many kinds of core-shell capsules, such as aminoplast shells (typically urea- or melamine-formaldehyde). Of particular interest are core-shell capsules in which the shell is made of a biodegradable material, such as gelatin.

In a further embodiment, the bio-bisabolol in encapsulated form may be incorporated into artificial fibres themselves at manufacture. This involves the addition of the capsules to the fibre material prior to spinning. Needless to say, the capsules should be such that a substantial proportion of them survive both the conditions of the melt and the extrusion process by which the fibres are formed, but the provision of such capsules is within the skill of the art and can be provided in each case by only routine experimentation.

Alternatively, there is the core-shell encapsulation method, in which the bio-bisabolol in pure or solution form is dispersed in an aqueous medium and a polymer shell formed around the individual disperse particles. The methods and materials used for the formation of such particles are well known to the art, typical polymeric materials being aminoplasts (such as urea- and melamine-formaldehyde resins), polyurea and acrylic resins. Particularly effective capsules are described in International publications WO 2016/207180 and WO 2017/001672.

The proportion of bio-bisabolol to be used, whether in free or in encapsulated form, will depend entirely on the nature of the product to which it will be added. The skilled person will be able to determine by non-inventive experimentation an appropriate proportion in every case. By way of typical example, the proportion will be from 0.1 ppm-5%, more particularly from 1 ppm-1%, by weight of the product, be it a laundry composition or a personal care composition. These proportions are given purely by way of example, and it may be possible in particular circumstances to use proportions outside of these.

In the case of wearable fabrics, the bio-bisabolol may be added to a laundry product or incorporated into the fibres themselves at manufactured, as hereinabove described. Alternatively, it may be applied to the fabric, or to the fibres constituting the fabric prior to weaving, by any convenient means, such as spraying or soaking. In the case of capsules, the capsules may be provided with a modifier, that is, a substance that has an affinity for the particular fibre material, and that fixes the capsules more securely to the fibre. Typical modifiers include non-ionic polysaccharides, such as mannan, glucan, glucomannan, xyloglucan, hydroxyalkyl cellulose, dextran, galactomannan, and mixtures thereof.

The result of the addition of bio-bisabolol produced by the method hereinabove described, when used on a fabric, results in a fabric that can be worn for long periods in contact with the skin with a considerable reduction in, and even complete lack of, skin redness, even in people who are sensitive to the residues of laundry products.

The disclosure is further described with reference to the following non-limiting examples, which describe particular embodiments.

### EXAMPLE 1

Bio-bisabolol was produced by the method described in Biochem. J. (2014) 463, 239-248, using Saccharomyces cerevisiae in a culture medium. The crude product was found to contain 65% (−)-α-bisabolol and less than 3% farnesol. This was purified by distillation to give (−)-α-bisabolol of about 99% purity and less than 0.10% farnesol.

### EXAMPLE 2

To a sample of a commercial fabric softener there is added 1% by weight of the bio-bisabolol of Example 1. 35 g of this fabric softener was added to a wash. The wash comprises cotton T-shirts.

An identical wash is carried out, but with the bisabolol absent from the fabric softener and used as a control.

An examination reveals that the washed fabric contained 249 μg of deposited bisabolol per g of fabric.

### EXAMPLE 3

### Use of Encapsulated Bio-Bisabolol.

Bio-bisabolol prepared according to Example 1 is added to a fragrance at 5% by weight. This fragrance is then encapsulated according to the method and using the material described in Example 1.3 of International Publication WO2008/098387. The result is a slurry of capsules, which slurry has a fragrance weight content of 36%, meaning that the bisabolol content in the slurry is 1.75% by weight.

The microcapsule slurry is added to a fabric softener at a rate of 1% by weight.

A fabric softener prepared in which the fragrance contains no bisabolol, but which is otherwise identical to that described above.

Both fabric softeners are subjected to identical washes, as described in Example 1 above. An examination of the fabric on which the fabric softener with the bisabolol is used shows that there is a deposition of 6 μg bisabolol per 1 g fabric.

### EXAMPLE 4

### Demonstration of Effect of Bio-Bisabolol on Fabric.

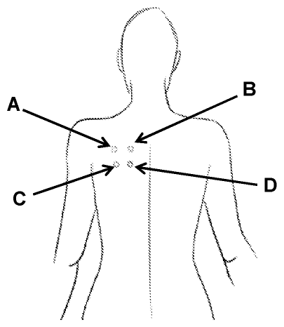

The T-shirts of Example 1, both those exposed to α-bisabolol according to Example 1 and the control, are worn for a continuous period of 24 hours by subjects. A number of measurements are taken, as detailed below. The measurements are taken at DO and D1 (T24h). FIG. 1 shows where on the body the measurements are taken.

The subjects consists of 40 volunteers (2 groups of 20) of minimum 18 years of age and who have dry skin and a tendency to an adverse reaction to softeners used on clothes with which they come 30 into extended contact.

Skin parameters measured during this study are:
Hydration (Corneometer® CM825 (C+K)) on the back of the subject. Three measurements are made on the back at position D in FIG. 1 at D0 and T24h.
TEWL (Tewameter® TM300 (C+K)) on the back of the subject at position B in FIG. 1—one measurement is made on the back at D0 and T24h
Color measure (Spectrocolorimeter® CM2500d (Konica Minolta)). This measures skin tone (L*) and redness (a*).

Three consistent measurements are made using MAV (Ø 8 mm) to take an average value for L* and a*. This is measured at position A of FIG. 1.
Analysis of the quantity of haemoglobin with illustrative photos (Siascope V®). One measurement is made on the back at position C in FIG. 1 at D0 and T24h. Illustrative photos are taken at D0 and T24h.
Self-assessment—a subjective evaluation of the efficacy by the subjects is done using a questionnaire

The following results are observed from the subjects with the T-shirts washed using the bisabolol-containing fabric softener in comparison with those subjects whose T-shirts were washed using unmodified fabric softener:
The colour measurements show a substantial average reduction in skin irritation as measured by the redness (a* parameter)
The TEWL measurement is reduced, indicating a restoration of skin barrier and a resulting increase in skin hydration. This correlates with a reduction in skin irritation and skin sensitivity.

### EXAMPLE 5

Comparison of the improvement in skin soothing effect using shampoos formulated with bio-bisabolol, chemically-produced bisabolol and plant-derived bisabolol.

Samples of the bio-bisabolol prepared according to Example 1, a commercially-available synthetic bisabolol and a bisabolol derived from chamomile oil were incorporated into shampoo formulations at 0.5% by weight. An untreated sample of the shampoo was included as placebo.

A double-blind, inter-individual and placebo-controlled clinical evaluation was performed on 80 volunteers (both men and women of age 18 and over). The subjects had Fitzpatrick skin types I to IV, and all suffered from pruritus of the scalp.

The volunteers washed and rinsed their hair once a day and itching intensity was evaluated on days 1 and 14, using a scale of 0-10, where 0 represents no itching and 10 represents no change in itching.

The results are shown in the table on the following page.

After 14 days of application, there was observed a significant reduction (of 25% at p<0.1) in scalp irritation for the bio-bisabolol treated group in comparison with the placebo group. The synthetic and plant bisabolol did not show any significant reduction in scalp irritation in comparison with the placebo. The enhanced soothing effect of the Example 1 bio-bisabolol was experienced by all the test volunteers.

| | Placebo | | | Example 1 bio-bisabolol | | | Synthetic bisabolol | | | Plant bisabolol | | |
|---|---|---|---|---|---|---|---|---|---|---|---|---|
| | Mean +/− | Δ % | | Mean +/− | Δ % | | Mean +/− | Δ % | | Mean +/− | Δ % | |
| | SEM | mean | p-value | SEM | mean | p-value | SEM | mean | p-value | SEM | mean | p-value |
| D0 | 4.1 ± 0.4 | | | 4.3 ± 0.4 | | | 4.4 ± 0.5 | | | 4.8 ± 0.5 | | |
| | | | | | | ns | | | ns | | | ns |
| D14 | 1.3 ± 0.3 | −66% | p < 0.0001 | 0.3 ± 0.2 | −93% | p < 0.0001 | 1.3 ± 0.4 | −70% | p < 0.0001 | 1.5 ± 0.4 | −69% | p = 0.0001 |
| | | | | | | (T0) | | | (T0) | | | (T0) |
| | | | | | | p < 0.1 | | | ns | | | ns |
| | | | | | | (placebo) | | | (placebo) | | | (placebo) |

1. A method of reducing skin irritation, comprising: the application of an α-bisabolol prepared by a biotechnological process to the skin.

2. The method of claim 1, in which the α-bisabolol is produced using Saccharomyces cerevisiae (baker's yeast) in a culture medium, followed by distillation.

3. The method of claim 1, in which the α-bisabolol is applied to a wearable fabric.

4. The method of claim 3, in which the α-bisabolol is applied by its addition to a laundry product in which the α-bisabolol has been incorporated.

5. The method of claim 1, in which the α-bisabolol is encapsulated.

6. The method of claim 3 in which the α-bisabolol is encapsulated and is incorporated into the fibres of the wearable fabric at their manufacture.

7. The method of claim 1, in which the α-bisabolol is applied to the skin by its incorporation into a skin application product.

8. The method of claim 7, in which the skin application product is a cosmetic product, a skin care product or a hair care product.

9. The method of claim 3, in which the proportion of α-bisabolol in the laundry product is from 0.1 ppm-5% by weight.

10. The method of claim 5, in which the α-bisabolol is the sole substance in the capsules.

11. The method of claim 5, in which the α-bisabolol forms part of an encapsulated fragrance.

12. A fabric washed with a laundry product and having a reduced potential for causing skin irritation on prolonged contact, the fabric having been washed using a laundry product comprising an irritation-reducing proportion of α-bisabolol.

13. A fabric comprising encapsulated α-bisabolol, the encapsulated α-bisabolol being incorporated into the fibres of the fabric at manufacture.

14. (canceled)

15. α-bisabolol of 99% purity, prepared by the method of claim 2.

16. The method of claim 7, in which the proportion of α-bisabolol in the skin application product is from 0.1 ppm-5% by weight.

17. The fabric of claim 12, wherein the α-bisabolol prepared by a biotechnological is produced by a biological process.

18. The fabric of claim 13, wherein the α-bisabolol prepared by a biotechnological is produced by a biological process.
